(19) United States
(12) Patent Application Publication
(10) Pub. No.: US 2002/0022239 A1
(43) Pub. Date: Feb. 21, 2002

(54) PHYTOFLUORS AS FLUORESCENT LABELS

(76) Inventor: JOHN CLARK LAGARIAS, DAVIS, CA (US)

Correspondence Address: LAW OFFICES OF JONATHAN ALAN QUINE, PO BOX 458, ALAMEDA, CA, 94501, US

(21) Appl. No.: 09/272,809

(22) Filed: Mar. 19, 1999

(51) Int. Cl. G01N 33/53

(52) U.S. Cl. 435/7.7

(57) ABSTRACT

This invention provides new fluorescent molecules useful for detection of target entities. In particular, it relates to fluorescent adducts comprising an apoprotein and a bilin.

### CROSS-REFERENCE TO RELATED APPLICATIONS

This is related to U.S. Ser. No. 08/904,871, filed Aug. 1, 1997 (also published as WO 98/04700), which is a continuation-in-part of U.S. Ser. No. 60/023,217, filed on Aug. 2, 1996, both of which are incorporated herein by reference for all purposes.

This work was supported by grants (MCB 92-06110, MCB 96-04511) from the National Science Foundation, a grant (GAM9503140) from the US Department of Agriculture, and by NIH training grant (5 T32 GM07377-17). The Government of the United States of America may have certain rights in the invention.

### FIELD OF THE INVENTION

The present invention relates to new fluorescent molecules useful for detection of target entities. In particular, it relates to fluorescent adducts comprising an apoprotein and a bilin.

### BACKGROUND OF THE INVENTION

The phytochromes comprise a family of biliprotein photoreceptors which enable plants to adapt to their prevailing light environment (Kendrick and Kronenberg (1994) Kendrick, Pp. 828 in Photomorphogenesis in Plants, Dordrecht, The Netherlands: Kluwer Academic Publishers). All phytochromes possess the ability to efficiently photointerconvert between red light absorbing Pr and far red light absorbing Pfr forms, a property conferred by covalent association of a linear tetrapyrrole (or bilin) with a large apoprotein. Phytochromes from cyanobacteria, to green algae and higher plants consist of a well conserved N-terminal polypeptide, roughly 390-600 amino acids in length (see FIG. 6 of WO 98/04700), to which the bilin prosthetic group phytochromobilin (PΦB) or phycocyanobilin (PCB) is bound.

The N-terminal domain of the phytochrome apoprotein is sufficient for spontaneous covalent attachment of ethylidene containing linear tetrapyrroles, a process requiring neither cofactors nor additional enzymes (Li et al. (1992) J. Biol. Chem., 267: 19204-19210). In higher plants, PΦB is bound to a conserved cysteine residue within the phytochrome apoprotein via a linkage identical to that found in the phycobiliprotein photosynthetic antennae of cyanobacteria, red algae and cryptomonads. The ability of the phytochrome photoreceptor to self assemble with its bilin prosthetic group contrasts with the phycobiliprotein photoreceptors which require separate enzymes for proper bilin attachment (Glazer (1989) J. Biol. Chem., 264: 1-4). Owing to the efficient photointerconversion between Pr and Pfr forms, phytochromes are poorly fluorescent molecules, unlike the phycobiliproteins which are intensely fluorescent and have been exploited as useful probes (see, e.g., U.S. Pat Nos. 4,857,474, and 4,520,110).

Fluorescent markers have found uses in molecular biology as labels for nucleic acid probes, antibodies, and other specific binding ligands in the detection of particular target moieties (e.g., particular nucleic acid sequences, receptors, etc.). Labeled binding molecules are used both in vitro and in vivo as diagnostic indicators and as research tools. Consequently there has been considerable interest and research on the development of fluorescent indicators.

Typically biological macromolecules (e.g., proteins or oligonucleotides) are labeled with a fluorescent marker (e.g., fluorescein, rhodamine, umbelliferone, and lanthanide chelates) either directly through a covalent linkage (e.g., a carbon linker), or indirectly whereby the macromolecule is bound to a molecule such as biotin or dioxigenin, which, is subsequently coupled to a fluorescently labeled macromolecular binding moiety (e.g., streptavidin or a labeled monoclonal antibody). Fluorescein and rhodamine are among the most commonly used fluorophore since they are readily available in an activated form for direct coupling to antigens or antibodies. Both fluorescein and rhodamines show good chemical stability and have a proven record in actual use as labels. However, macromolecules labeled with these fluorophores suffer from chemical quenching of fluorescence, and it is difficult to control the labeling of discrete sites within the macromolecule.

These fluorescent labeling systems also suffer the disadvantage that the fluorescent complexes and/or their binding moieties are relatively large, and must be prepared and supplied from an exogenous source because most organisms are not capable of synthesizing these molecules. In addition, these molecules are often toxic to the subject organism.

With only one exception, the Green Fluorescent Protein (GFP) from the jellyfish Aequorea victoria (U.S. Pat No. 5,491,084), the ability to synthesize a fully functional fluorescent macromolecule has been restricted to the host organism in which the protein naturally occurs. Because the nucleic acid encoding GFP can be cloned into a cell and expressed to yield a non-fluorescent protein precursor that spontaneously assembles its own fluorophore, GFP has gained widespread utility as a selectable marker and a probe of cellular events (Cubitt et al. (1995) Trends In Biochem. Sci. 20, 448-455). From many attempts to improve the properties of GFP through genetic engineering, it is clear that there is a finite spectral window within which GFP is useful as a fluorescent marker. The development of additional protein-based fluorescent markers that can be functionally expressed in various cell types by standard genetic engineering techniques with an extended fluorescence wavelength range, and a variety of useful biochemical properties is desirable.

A recent development in the field of fluorescent labeling has been the use of phycobiliprotein conjugates. Phycobiliproteins are a class of highly fluorescent proteins that form a part of the light-harvesting system in the photosynthetic apparatus of bluegreen bacteria and of two groups of eukaryotic algae, red algae and the cryptomonads. A particularly useful variation of their use comprises preparation of a phycobiliprotein tandem conjugate with a large Stokes shift. An example of such a conjugate is the covalent attachment of the phycobiliproteins, phycoerythrin and allophycocyanin. The resulting tandem conjugate has a large Stokes shift with an emission maximum at 660 nm and an excitation waveband that starts at about 440 nm. However, production of such tandem complexes requires human intervention in the formation of a covalent or other chemical bond between the two components, therefore increasing the complexity of the production of the final conjugate.

Despite these advances, the art fails to provide fluorescent markers that can be easily produced and readily engineered to provide strong fluorescent signals over a wide range of wavelengths. The present invention addresses these and other needs.

### SUMMARY OF THE INVENTION

This invention provides a new class of fluorescent protein adducts (phycobilin conjugates) that are generally suitable for use as fluorescent markers. Owing to their long wavelength absorption maxima, their high molar absorption coefficients and the ability of recombinant phytochrome apoproteins to spontaneously assemble with a variety of bilin chromophore precursors, the phytochromes are potentially ideal fluorescent markers.

Phytochromes perform a key role as light sensors in most photosynthetic organisms, via photoisomerization of the covalently bound phytochromobilin or phycocyanobilin prosthetic group which induces a protein conformational change and subsequent signal transduction cascade. The adduct between recombinant apophytochrome and phycoerythrobilin (PEB), the natural chromophore precursor of phycoerythrin, is highly fluorescent because it lacks the double bond required for photoisomeration. This invention demonstrates that fluorescent apophytochrome-bilin conjugates (e.g., apophytochrome-PEB adducts), which are referred to herein as the “phytofluors”, are intensely fluorescent, photostable proteins useful as probes for biological research.

In a preferred embodiment, the fluorescent adducts (i.e., phytofluors) of this invention comprise a protein component (an apoprotein) and a nitrogen heterocyclic compound (e.g., a polypyrrole). In a preferred embodiment the nitrogen heterocycle is a dipyrrole, tripyrrole, tetrapyrrole, or analogues thereof, with linear tetrapyrroles and analogues thereof being most preferred. In some embodiments, higher order pyrroles and their analogues can also be used. One particularly preferred bilin is phycoerythrobilin (PEB). The apoprotein is preferably an apophytochrome or analogue thereof. Preferred analogues are recognized by and thus comprise the consensus sequence discussed above. The apoprotein can be derived from vascular and non-vascular plants, green alga, bacteria or cyanobacteria, or can be chemically synthesized de novo. Thus, preferred apoproteins are encoded by plant genes, algal genes, bacterial genes, or cyanobacterial genes. Particularly preferred apoproteins include any of the apoproteins described herein or those listed in the sequence listing or conservative substitutions of these sequences, while most preferred apoproteins include apoproteins from plants (e.g., oats with an apoprotein having about 1100 amino acid residues), green algae (e.g., Mesotaenium caldariorum), or cyanobacteria (as illustrated in the sequence listing), or related, proteins having conservative substitutions. Truncated apoproteins consisting of a chromophore domain; the apoprotein N-terminal subsequence sufficient for lysase activity are particularly preferred. One preferred N-terminal subsequence consists of less than about 600 N-terminal amino acids, more preferably less than about 400 N-terminal amino acids, and most preferably about 200 N-terminal amino acids.

In one preferred embodiment, this invention provides for a moiety that is labeled with one or more of the fluorescent adducts of this invention. The fluorescent adduct is attached covalently, or non-covalently, directly, or through a linker to a moiety that is to be labeled. The moiety can be virtually any composition, including for example, a biological molecule (biomolecule), an organelle, a cell, a tissue, virtually any naturally occurring natural or synthetic material that is chemically compatible with the fluorescent adduct, and even an article of manufacture. In a particularly preferred embodiment, the fluorescent adducts of this invention are attached to biological molecules including, but not limited to proteins, carbohydrates, lipids, and nucleic acids. Particularly preferred biological molecules are members of binding pairs (binding partners) that specifically bind to a target molecule. Preferred members of binding pairs include antibodies, nucleic acids, lectins, enzymes, ligands, receptors, and the like.

The fluorescent adduct can be joined to the moiety to be labeled either by attachment to the bilin or by attachment to the apoprotein, with attachment to the apoprotein being most preferred. The apoprotein can be chemically conjugated to the subject (labeled) molecule or, where the subject moiety is a protein or contains a protein component, the apoprotein can be fused to the amino or carboxyl terminus of the protein or protein component through a peptide bond thereby forming a fusion protein. The fusion protein can also be a recombinantly expressed fusion protein. Alternatively, the apoprotein can be joined to the protein or protein component of the subject moiety through linkages between side chains (e.g., a disulfide linkage between cysteines).

This invention also provides methods of use for the above-described fluorescent adducts and for the compositions comprising a moiety joined to any of the fluorescent adducts described above or herein. Thus, for example, in one embodiment, this invention provides for a method of testing the presence of a biomolecule in a sample. The method involves providing a sample comprising a biomolecule linked to a fluorescent adduct consisting of an apoprotein and a bilin chromophore and contacting the sample with light which causes the fluorescent adduct to emit light, and detecting the emitted light thereby detecting the presence of the biomolecule. In one particularly preferred embodiment, the sample is contacted with light having a wavelength of about 570 nm. The step of detecting the emitted light may include detecting light having a wavelength of about 590 nm. In a particularly preferred embodiment, the biomolecule is one or more of any of the above-identified biomolecules.

This invention also provides methods of expressing and detecting a selectable marker. These methods include providing a nucleic acid that encodes a protein of interest and any of the apoproteins described above and herein. The expressed apoprotein is contacted with a bilin, more preferably one of the bilins described above or herein to form a fluorescent adduct. Finally, the fluorescent adduct is contacted with light which causes the fluorescent adduct to fluoresce emitting light which is then detected thereby indicating the presence of the selectable marker.

In still yet another embodiment, this invention provides a method of detecting and/or quantifying protein-protein interactions. The two subject proteins are expressed in fusion with or conjugated to an apoprotein. The apoproteins are selected such that, when combined with their respective bilins, they form a first and a second fluorescent adduct, respectively. The first adduct fluoresces at a wavelength absorbed by the second adduct which then emits at a different wavelength. Exposure of the proteins with light causes the first fluorescent adduct to emit light that is transferred to the second fluorescent adduct which then emits light at a different wavelength thereby indicating that the two proteins are in close proximity. This invention also provides for numerous other variants of this assay which are disclosed herein.

### DEFINITIONS

The term “fluorescent adduct” refers to a fluorescent molecule (i.e., one capable of absorbing light of one wavelength and emitting light of a second wavelength) comprising an “apoprotein” (also referred to as an apophytochrome) component joined to a “bilin” component, both of which are described below. The fluorescent phytochrome-bilin conjugates (e.g., phytochrome-PEB adducts), are also referred to herein as “phytofluors”. The manner in which the two components are joined to form an adduct is irrelevant to the present invention. Typically, the two components spontaneously form an adduct through covalent interactions. The components may also be deliberately linked through covalent bonds (e.g., through the use of crosslinking reagents). The fluorescent adducts of this invention do not require pairing of an apoprotein with its corresponding native bilin. To the contrary, the invention contemplates adducts consisting of naturally occurring or engineered apoproteins with bilins derived from different organisms, or with non-naturally occurring synthetic linear pyrroles.

The terms “apoprotein”, “apophytochrome”, or “apoprotein polypeptide”, as used herein, refer to polypeptides derived from eukaryotes, such as vascular plants, non-vascular plants, and algae, or from prokaryotes, such as cyanobacteria, or other eubacteria and archaebacteria. The term encompasses both naturally occurring apoproteins and variant polypeptides derived through mutagenesis. The apoproteins have a hydrophobic pocket, referred to as chromophore or bilin binding site, capable of forming an adduct with a bilin component. The prototypical eukaryotic apoproteins of the invention are typically homodimeric proteins about 1100 amino acids in length, each subunit being composed of two major domains. The globular 70 kD N-terminal domain contains the hydrophobic pocket, while the more elongated 55 kD carboxyl terminal domain contains the sites at which the two subunits are associated. Apophytochromes containing a bilin binding site can be readily identified by one of skill in the art by comparison of the polypeptide sequence in question with the apophytochrome consensus sequence discussed above using standard sequence comparison methodologies. For a general discussion of apoprotein structure and function, see, Quail et al. (1997) in Plant Cell and Environment, 20: 657-665.

The preferred apoproteins of the invention typically consist essentially of a chromophore domain. The terms “chromophore domain” or “minimal chromophore domain” or “lyase domain” refer to the apoprotein N-terminal subsequence sufficient for lyase activity and thereby form a covalent bond between the apoprotein and the bilin. Lyases are enzymes that catalyze the reversible formation of a covalent adduct between a hydroxyl- or thiol-containing substrate and a substrate containing a double bond (i.e. addition of a nucleophile to a double bond). Chromophore domains are typically between about 180 and about 250 amino acids, typically between about 190 amino acids and about 220 amino acids, and usually about 200 amino acids in length (e.g., 197 amino acids). Typically, this spontaneous assembly results in the formation of a phytofluor.

The apoproteins of the invention typically comprise less than about 600 amino acids of the N terminus (including the chromophore or lyase domain) of the full length apoprotein, preferably less than about 515 amino acids, more preferably less than about 450 amino acids and most preferably less than about 400, 390, or even 350 N-terminal amino acids. Preferred apoproteins of the invention typically comprise between about 200 and about the 400 N-terminal amino acids of the full length apoprotein, including the lyase domain. A preferred embodiment consists essentially of the lyase domain.

The “bilin” components of the adducts of the invention are linear polypyrroles (e.g., di-, tri-, or tetrapyrroles) capable of fluorescing when associated with an apoprotein. Typically, the bilin components of the invention are isolated from vascular plants, algae, or cyanobacteria according to standard techniques. The bilin components can also be synthesized de novo. For a general discussion of bilins useful in the present invention see, Falk (1989) Pp. 355-399 in: The Chemistry of Linear Oligopyrroles and Bile Pigments. pp 355-399. Springer-Verlag, Vienna.

The phrase “nucleic acid” refers to a single or double-stranded polymer of deoxyribonucleotide or ribonucleotide bases read from the 5′ to the 3′ end. It includes cDNA, self-replicating plasmids, infectious polymers of DNA or RNA and non-functional DNA or RNA.

The term “subsequence” when referring to a nucleic acid refers to a nucleic acid sequence that comprises a part of a longer sequence of a nucleic acid, and when referring to a peptide refers to an amino acid sequence that comprises part of a longer sequence of a peptide, polypeptide or protein.

Two nucleic acid sequences or polypeptides are said to be “identical” if the sequence of nucleotides or amino acid residues, respectively, in the two sequences is the same when aligned for maximum correspondence as described below. The term “complementary to” is used herein to mean that the complementary sequence is identical to all or a portion of a reference polynucleotide sequence.

Sequence comparisons between two (or more) polynucleotides or polypeptides are typically performed by comparing sequences of the two sequences over a “comparison window” to identify and compare local regions of sequence similarity. A “comparison window”, as used herein, refers to a segment of at least about 20 contiguous positions, usually about 50 to about 200, more usually about 100 to about 150 in which a sequence may be compared to a reference sequence of the same number of contiguous positions after the two sequences are optimally aligned.

Optimal alignment of sequences for comparison may be conducted by the local homology algorithm of Smith and Waterman Adv. Appl. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85: 2444 (1988), by computerized implementations of these algorithms such as CLUSTALW, GAP, BESTFIT, BLAST, FASTA, and TFASTA (Wisconsin Genetics Software Package, Genetics Computer Group (GCG), 575 Science Dr., Madison, Wis.), or by inspection.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has at least 60% sequence identity, preferably at least 80%, more preferably at least 90% and most preferably at least 95%, compared to a reference sequence using the programs described above preferably BLAST) using standard parameters. In one embodiment, 25% sequence identity over a window of 200 amino acids coupled with information regarding the apophytochrome consensus sequence is sufficient to identify a new apophytochrome. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 40%, preferably at least 60%, more preferably at least 90%, and most preferably at least 95%. Polypeptides which are “substantially similar” share sequences as noted above except that residue positions which are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine.

Another indication that nucleotide sequences are substantially identical is if two nucleic acid molecules hybridize to each other, or to a third nucleic acid, under stringent conditions. Stringent conditions are sequence dependent and will be different in different circumstances. Generally, stringent conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched complementary nucleic acid sequence. Typically, stringent conditions will be those in which the salt concentration is about 0.02 molar at pH 7 and the temperature is at least about 60° C. Stringent conditions for a standard Southern hybridization will include at least one wash (usually 2) in 0.2X SSC at a temperature of at least about 50° C., usually about 55° C., for 20 minutes, or equivalent conditions.

The term “conservative substitution” is used herein to refer to replacement of amino acids in a protein with different amino acids that do not substantially change the functional properties of the protein. Thus, for example, a polar amino acid might be substituted for a polar amino acid, a non-polar amino acid for a non-polar amino acid, and so forth. The following six groups each contain amino acids that are conservative substitutions for one another:

1)Alanine (A), Serine (S), Threonine (T);

2) Aspartic acid (D), Glutamic acid (E);

3)Asparagine (N), Glutamine (Q);

4)Arginine (R), Lysine (K);

5)Isoleucine (I), Leucine (L), Methionine (M), Valine (V); and

6) Phenylalanine (F), Tyrosine (Y), Tryptophan (W).

A biological “binding partner” or a member of a “binding pair” refers to molecules that specifically bind other molecules to form a binding complex such as antibody-antigen, lectin-carbohydrate, nucleic acid-nucleic acid, biotin-avidin, etc.

The term “specifically binds”, as used herein, when referring to a biomolecule (e.g., protein, nucleic acid, antibody, etc.), refers to a binding reaction which is determinative of the presence of a specific biomolecule within a heterogeneous population of proteins and/or other biologics. Thus, under designated conditions (e.g. immunoassay conditions in the case of an antibody), the specified ligand or antibody binds to its particular “target” biomolecule (e.g. a receptor protein) and does not bind in a significant amount to other proteins or other biomolecules present in the sample, or to other proteins or other biomolecules with which the ligand or antibody may come in contact in an organism.

The term “antibody”, as used herein, includes various forms of modified or altered antibodies. Such forms include, but are not limited to, an intact immunoglobulin, an Fv fragment containing only the light and heavy chain variable regions, an Fv fragment linked by a disulfide bond (Brinkmann, et al. (1993) Proc. Natl. Acad. Sci. USA, 90: 547-551), an Fab or (Fab)′₂ fragment containing the variable regions and parts of the constant regions, a single-chain antibody and the like (Bird et al. (1988) Science 242: 424-426; Huston et al. (1988) Proc. Nat. Acad. Sci. USA 85: 5879-5883). The antibody may be of animal (especially hamster, mouse, rat, rabbit, pig, or goat) or human origin or may be chimeric (Morrison et al., Proc Nat. Acad. Sci. USA 81: 6851-6855 (1984)) or humanized (Jones et al. (1986) Nature 321: 522-525, and published UK patent application No: 8707252). Methods of producing antibodies suitable for use in the present invention are well known to those skilled in the art and can be found described in such publications as Harlow & Lane, Antibodies: A Laboratory Manual, Cold Spring Harbor Laboratory (1988), and Asai, Methods in Cell Biology Vol 377: Antibodies in Cell Biology, Academic Press, Inc. N.Y. (1993).

### BRIEF DESCRIPTION OF THE DRAWINGS

FIG. 1 shows the putative structural organization of the predicted protein products of these open reading frames. The roughly 200 amino acid chromophore domain of these proteins which include the cysteine site of bilin attachment is shown with an asterisk.

FIG. 2 shows a multiple sequence alignment of the chromophore domains of representative eukaryotic phytochromes the Arabidopsis (At) phyA, phyB/D, phyC and phyE proteins and the green algal phytochrome (Mcphy1b) and the cyanobacterial phytochrome sequences of the invention, cph1-2 and cp11-6. In the figure, each cyanobacterial protein is referred to by its cyanobase locus designation. The correspondence between protein name and locus name is as follows:

| | |
|---|---|
| Cph1 | SLR0473 |
| Cph2 | SLL0821 |
| Cpl1 (Cph3) | SLL1473 |
| Cpl2 (Cph4) | SLL1124 |
| Cpl3 (Cph5) | SLL0041 |
| Cpl4 (Cph6) | SLR1212 |
| Cpl5 (Cph7) | SLR1393 |
| Cpl6 (Cph8) | SLR1969 |

The comparison was obtained using the Wisconsin Genetics Computing Group program PILEUP. This multiple sequence alignment defines the bilin chromophore binding domain of the phytochrome superfamily.

FIG. 3A shows the expression vector used to express a Strep-Tagged version of N-terminal 197 amino acid region of Cph2 (Cph2-N197).

FIG. 3B provides the sequence of Cph2-N197 (SEQ ID NO: 9).

### DETAILED DESCRIPTION

This invention is directed to fluorescent adducts, referred to herein as phytofluors, and their use as fluorescent markers or labels in a variety of contexts. The phytofluors comprise an apoprotein component (e.g. an oat or cyanobacterial apophytochrome) joined to a bilin component (e.g., phycoerythrobilin (PEB)). The phytofluors (fluorescent adducts) may be chemically conjugated or fused (i.e. recombinantly expressed as a fusion protein) to a subject moiety that is to be so labeled. In a preferred embodiment the labeled moiety is a member of a biological binding pair for use in any known or later discovered technique involving fluorescent labeling of analytes or other moieties.

The apoproteins and bilins forming the fluorescent phytofluors of this invention are available from natural sources or can be modified to provide novel complexes having different absorbance, emission, or labeling characteristics. These compositions find use for labeling of virtually any molecule or material that is chemically compatible with the fluorescent adducts. The phytofluors are well suited for labeling biological molecules and are particularly used to label a biochemical binding-pair member so that the resulting conjugates or fusions can be used in assays involving non-covalent binding to the complementary member of the specific binding pair. A wide variety of methods involve competitive or non-competitive binding of ligand to receptor for detection, analysis, or measurement of the presence of ligand or receptor.

Thus, for example, in one embodiment, this invention provides for antibodies or antibody fragments to which the fluorescent adducts (phytofluors) of this invention are joined (either covalently or non-covalently). The antibodies are capable of specifically binding to the antigen to which they are directed. Detection of the presence, absence, or amount of fluorescence of the antibody-bound fluorescent adduct of this invention provides an indication of presence, absence, or amount of analyte to which the antibody is directed.

Similarly phytofluor labeled antibodies, or other ligands, can be used in immunohistochemical applications. In this context, fluorescent adduct labeled antibodies are used to probe cells, tissues, and sections thereof. When the subject sample is contacted with the labeled ligand, the ligand binds and localizes to specific regions of the sample in which the target molecule (the molecule or moiety recognized by the ligand) is located. Localization and/or quantification of the fluorescent signal produced by the attached phytofluor provides information concerning the location and/or quantity of the target molecule in the sample. One of skill in the art will appreciate that the phytofluors of this invention are also well suited for in situ and in vivo labeling of molecules, cells, and cellular components.

The phytofluor labels of this invention can be attached to a wide variety of biological molecules in addition to antibodies. This may include proteins, in particular proteins recognized by particular antibodies, receptors, enzymes, or other ligands, nucleic acids (e.g., single or double stranded DNA, cDNA, mRNA, cRNA, rRNA, tRNA, etc.) various sugars and polysaccharides, lectins, enzymes, and the like. Uses of the various labeled biomolecules will be readily apparent to one of skill in the art. Thus, for example, labeled nucleic acids can be used as probes to specifically detect and/or quantify the presence of the complementary nucleic acid in, for example, a Southern blot.

The phytofluors of this invention can be attached to non-biological molecules and various articles of manufacture. Thus, for example where it is desired to associate an article of manufacture with a particular manufacturer, distributor, or supplier, the phytofluor, or simply one component of the phytofluor can be attached to the subject article. Later development (e.g., by addition of the second component such as bilin or apoprotein) and exposure to an appropriate light source will provide a fluorescent signal identifying the article as one from a source of such labeled articles.

In another embodiment, the phytofluors of this invention can be used for probing protein-protein interactions. In a preferred embodiment, two apoprotein cDNA constructs are used. The first construct will encode a apoprotein species whose assembly with a given bilin emits at a well defined wavelength (donor). The second construct will encode an apoprotein species whose assembly with the same, or different, bilin produces a fluorescent species that both absorbs and emits light to longer wavelengths (acceptor). Protein-protein interaction between two proteins of interest (e.g., protein X and protein Y) is identified following their co-expression as translational fusions with apoprotein in constructs 1 (donor) and 2 (acceptor) using fluorescence energy transfer from the shorter wavelength-absorbing donor species to the longer wavelength-absorbing acceptor species. In a preferred embodiment, the fluorescent phytochrome species are selected to have good spectral overlap. Proximity caused by the protein-protein interaction between the translational fused proteins X and Y will then permit fluorescence energy transfer thereby providing an indication of proximity between protein X and protein Y. This application can utilize the uptake of exogenous bilin pigment into living cells, or alternatively, may use endogenously expressed bilins in various organisms and cell types.

In a specific application, a yeast or E. coli strain containing donor construct 1, engineered to produce a fluorescent chimeric protein bait with a known cDNA sequence, will be co-transformed, simultaneously or sequentially, with a prey cDNA library (i.e., plasmid or phage). The prey cDNA library will be constructed using acceptor construct 2 for expression of apoprotein-protein fusions which yield fluorescent tagged protein products in the presence of the correct bilin. Co-transformation events which express prey proteins in the library that interact with the expressed bait polypeptide can be identified by illuminating the shorter wavelength absorbing donor phytofluor species and viewing emission from the longer wavelength acceptor phytofluor emitting species. Actinic illumination for this screen can either be obtained with a quartz halogen projector lamp filtered through narrow bandpass filters or with a laser source and fluorescence detection of colonies using digital imaging technology (Arkin et al. (1990) Bio-Technology 8: 746-749). Fluorescent activated cell sorting (FACS) can also be used to identify cells co-expressing interacting donor and acceptor proteins.

In another application of this invention, the apoprotein cDNA in donor construct 1 “prey” is substituted with a green fluorescent protein (GFP) cDNA or construction of GFP-tagged cDNA expression libraries. By co-expression of apoprotein- tagged bait construct (Construct 2 above) with the GFP-tagged “prey” library, proteins which interact with the bait polypeptide will be visualized by energy transfer from GFP to the phytochrome tagged bait using, for example, digital imaging technology or FACS. The ability of GFP to spontaneously assemble its fluorophore makes it unnecessary to make two apoprotein constructs which have different fluorescence properties.

In a third specific application, chimeric apoprotein-protein X cDNA (where protein X is any protein of interest) are expressed in transgenic eukaryotes (yeast, plants, Drosophila, etc.) in order to study the subcellular localization of protein X in situ. Following feeding of exogenous bilin, subcellular localization can be performed using fluorescence microscopy (e.g., laser confocal microscopy).

In one particularly preferred embodiment, the phytofluors of this invention are used as in vitro or in vivo labels in a manner analogous to the use of Green Fluorescent Protein (GFP). This typically involves transfecting a cell with a nucleic acid encoding an apoprotein in such an manner that the cell expresses the apoprotein (e.g., the nucleic acid is a component of an expression cassette). When the apoprotein is contacted with the appropriate bilin, supplied either exogenously or produced endogenously, the phytofluor (fluorescent adduct) self assembles and thereby produces a fluorescent marker.

Uses of such a marker are well known to those of skill in the art (see, e.g., U.S. Pat. No. 5,491,084 which describes uses of GFP). In one preferred embodiment, the phytofluor can be used as a marker to identify transfected cells. In the simplest approach, a nucleic acid expressing an apoprotein such as that described in Example 1 can be provided as a marker in a vector. The apoprotein, along with the cloned protein of interest, will be expressed in the transfected host. Application of the appropriate exogenous bilin will cause formation of the fluorescent adduct permitting ready detection of the transformed cell. Alternatively, the apoprotein can form an adduct with an endogenous bilin produced by the transformed organism (e.g., a plant cell). In this embodiment, the apoprotein will be a variant which forms fluorescent adduct when combined with the naturally occurring bilin.

Based on the disclosure provided herein, one of skill will readily appreciate that there are numerous other uses to which the phytofluors (fluorescent adducts) of this invention can be applied.

Preparation of apoprotein polypeptides.

Apoprotein polypeptides used in the phytofluors of this invention can be expressed recombinantly or isolated from natural sources according to standard techniques. The polypeptides or nucleic acids encoding them can be prepared from a wide range of organisms including vascular plants, algae, and cyanobacteria.

In higher plants, apoprotein polypeptides are encoded by a gene family of at least five structurally related members designated PHYA-PHYE (see, Terry et al. (1993) Arch. Biochem. Biophys. 306:1-15 and Scharrock et al. (1989) Genes Dev. 3:1745-1757). The primary structures of all apoproteins are very similar, with a polypeptide of about 1100 amino acids in length (Quail et al. in Phytochrome Properties and Biological Action (Thomas and Johnson eds.) pp 13-38, (Springer-Verlag, Berlin 1991)). The native protein is a homodimer; the individual subunits being composed of two major domains. The globular 70 kD N-terminal domain contains the hydrophobic pocket in which the bilin chromophore resides (Gabriel et al. (1993) J. Theor. Biol. 44:617-645. The more elongated 55 kD carboxyl terminal domain contains the sites at which the two subunits are associated (Edgerton et al. (1992) Plant Cell 4:161-171). This domain is also responsible for phytochrome function, although both domains are thought to participate in the signal transmission process in native phytochrome.

Phytofluor apoproteins can be isolated from natural sources, most preferably from bilin-deficient natural sources including, vascular and nonvascular plants, algae and cyanobacteria using standard protein isolation techniques well known to those of skill in the art. Generally, these methods involve standard techniques well known in the art, including selective precipitation with such substances as ammonium sulfate, column chromatography, immunopurification methods, and others. See, for instance, R. Scopes, Protein Purification: Principles and Practice, Springer-Verlag: New York (1982).

In preferred embodiments, the polypeptides are produced recombinantly. Standard methods for preparation of recombinant proteins can be used for this purpose. For a discussion of the general laboratory procedures required for this purpose see, Sambrook, et al., (1989) Molecular Cloning: A Laboratory Manual (2nd Ed.), Vol. 1-3, Cold Spring Harbor Laboratory, Cold Spring Harbor, N.Y..

Nucleic acids encoding apoprotein polypeptides can be isolated from a number of organisms according to standard techniques. Exemplary genes are those isolated from higher plants (e.g., AsphyA and AtphyA), and the green alga Mesotaenium caldariorum (i.e. Mcphy1b). In addition, genes encoding apophytochrome can be obtained from cyanobacteria. It was a discovery of this invention that the cyanobacteria Synechocystis sp. produces an apophytochrome. In particular, the open reading frame listed in GenBank D64001, locus 1001165 and designated herein as S6803phyl was determined to be an apophytochrome by sequence alignment methods. Having identified herein that cyanobacteria produce apophytochromes, identification of other cyanobacterial apophytochromes can be accomplished using routine methods available to one of skill in the art. Sequences for these apoproteins are provided in the sequence listing below. The corresponding nucleic acid sequences are known to those of skill in the art. One of skill will recognize that these sequences can be used to determine the design primers and probes for isolation of related genes in other organisms. Cyanobacterial nucleic acid sequences are also available at the Cyanobase Web Site. http://www.kazusa.or.jp/cyano/.

Generally, recombinant expression techniques involve the construction of recombinant nucleic acids and the expression of genes in transfected cells. Molecular cloning techniques to achieve these ends are known in the art. A wide variety of cloning and in vitro amplification methods suitable for the construction of recombinant nucleic acids are well-known to persons of skill. Examples of these techniques and instructions sufficient to direct persons of skill through many cloning exercises are found in Berger and Kimmel, Guide to Molecular Cloning Techniques, Methods in Enzymology volume 152 Academic Press, Inc., San Diego, Calif. (Berger); and Current Protocols in Molecular Biology, F. M. Ausubel et al., eds., Current Protocols, a joint venture between Greene Publishing Associates, Inc. and John Wiley & Sons, Inc., (1994 Supplement) (Ausubel).

The polypeptides are expressed in a recombinantly engineered cell such as plants, bacteria, yeast, insect (especially employing baculoviral vectors), and mammalian cells. It is expected that those of skill in the art are knowledgeable in the numerous expression systems available for expression of the DNA encoding apoprotein polypeptides. No attempt to describe in detail the various methods known for the expression of proteins in prokaryotes or eukaryotes will be made.

In brief, the expression of natural or synthetic nucleic acids encoding the polypeptides will typically be achieved by operably linking the DNA or cDNA to a promoter (which is either constitutive or inducible), followed by incorporation into an expression vector. The vectors can be suitable for replication and integration in either prokaryotes or eukaryotes. Typical expression vectors contain transcription and translation terminators, initiation sequences, and promoters useful for regulation of the expression of the DNA encoding the binding domains. To obtain high level expression of a cloned gene, it is desirable to construct expression plasmids which contain, at the minimum, a strong promoter to direct transcription, a ribosome binding site for translational initiation, and a transcription/translation terminator.

Expression in Prokaryotes

Examples of regulatory regions suitable for this purpose in E. coli are the promoter and operator region of the E. coli tryptophan biosynthetic pathway as described by Yanofsky (1984) J. Bacteriol., 158: 1018-1024 and the leftward promoter of phage lambda (P_{L}) as described by Herskowitz and Hagen (1980) Ann. Rev. Genet., 14: 399-445. The inclusion of selection markers in DNA vectors transformed in E. coli is also useful. Examples of such markers include genes specifying resistance to ampicillin, tetracycline, or chloramphenicol. See Sambrook et al. for details concerning selection markers for use in E. coli.

Expression systems for expressing the polypeptides are available using E. coli, Bacillus sp. (Palva et al. (1983) Gene 22:2 29-235; Mosbach et al. Nature, 302:543-545) and Salmonella. E. coli systems are preferred.

The apoprotein polypeptides produced by prokaryote cells may not necessarily fold properly. During purification from E. coli, the expressed polypeptides may first be denatured and then renatured. This can be accomplished by solubilizing the bacterially produced proteins in a chaotropic agent such as guanidine HCl and reducing all the cysteine residues with a reducing agent such as beta-mercaptoethanol. The polypeptides are then renatured, either by slow dialysis or by gel filtration (see, e.g., U.S. Pat. No. 4,511,503).

Expression in Eukaryotes

A variety of eukaryotic expression systems such as yeast, insect cell lines and mammalian cells, are known to those of skill in the art. As explained briefly below, the apoprotein polypeptides may also be expressed in these eukaryotic systems.

Expression in Yeast

Synthesis of heterologous proteins in yeast is well known and described. Methods in Yeast Genetics, Sherman et al., Cold Spring Harbor Laboratory, (1982) is a well recognized work describing the various methods available to produce the polypeptides in yeast.

Preferred yeast expression systems are described in Wahleithner et al. (1991) Proc. NatL. Acad. Sci. USA 88:10387-10391, Murphy and Lagarias (1997) Photochem. Photobiol., 65: 750-758, and Wu et al. (1996) Proc. Natl. Acad. Sci., USA, 93: 8989-8994. Further examples of yeast expression are described below. A number of yeast expression plasmids like YEp6, YEp 13, YEp4 can be used as vectors. A gene of interest can be fused to any of the promoters in various yeast vectors. The above-mentioned plasmids have been fully described in the literature (Botstein et aL (1979) Gene, 8: 17-24; Broach et al. (1979) Gene, 8: 121-133).

The polypeptides can be isolated from yeast by lysing the cells and applying standard protein isolation techniques to the lysates. The monitoring of the purification process can be accomplished by using spectroscopic techniques, or by using Western blot techniques or radioimmunoassays, or other standard immunoassay techniques.

Expression in Plants

The apoprotein polypeptides of this invenion can also be expressed in plants or plant tissues. Plant tissue includes differentiated and undifferentiated tissues of plants including, but not limited to, roots, shoots, leaves, pollen, seeds, tumor tissue, such as crown galls, and various forms of aggregations of plant cells in culture, such as embryos and calli. The plant tissue may be in plants, cuttings, or in organ, tissue, or cell culture.

The recombinant DNA molecule encoding the apoprotein polypeptide under the control of promoter sequences may be introduced into plant tissue by any means known to the art. The technique used for a given plant species or specific type of plant tissue depends on the known successful techniques. The various DNA constructs described above may be introduced into the genome of the desired plant by a variety of conventional techniques. For example, the DNA construct may be introduced directly into the genomic DNA of the plant cell using polyethylene glycol precipitation (Paszkowski et al. (1984) Embo J. 3: 2717-2722) electroporation and microinjection of plant cell protoplasts (Fromm et al. (1985) Proc. Natl. Acad. Sci. USA 82: 5824), or the DNA constructs can be introduced into plant tissue using ballistic methods, such as DNA particle bombardment (Klein et al. (1987) Nature 327: 70-73). Alternatively, the DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker gene(s) (if present) into the plant cell DNA when the cell is infected by the bacteria. For a review of gene transfer methods for plant and cell cultures see, Fisk et al. (1993) Scientia Horticulturae 55: 5-36 (1993) and Potrykus (1990) CIBA Found. Symp. 154: 198.

Agrobacterium tumefaciens-meditated transformation techniques are the most commonly used techniques for transferring genes into plants. These techniques are well described in the scientific literature. See, for example Horsch et al. (1984) Science 233: 496-498, Fraley et al. (1983) Proc. Natl. Acad. Sci. USA 80: 4803, and Hooykaas (1989) Plant Mol. Biol 13: 327-336, Bechtold et al. (1993). Comptes Rendus De L Academie Des Sciences Serie Iii-Sciences De La Vie-Life Sciences 316: 1194-1199, Valvekens et al. (1988) Proc. Natl. Acad. Sci. USA 85: 5536-5540.

All species which are natural plant hosts for Agrobacterium are transformable in vitro. Most dicotyledonous species can be transformed by Agrobacterium. Monocotyledonous plants, and in particular, cereals, have not previously been regarded as natural hosts to Agrobacterium. There is, however, growing evidence that monocots can be transformed by Agrobacterium. Using novel experimental approaches cereal species such as rye (de la Pena et al. (1987) Nature 325: 274-276), corn (Rhodes et al. (1988) Science 240: 204-207), and rice (Shimamoto et al., (1989) Nature 338: 274-276) may now be transformed.

Transformation of a number of woody plants using Agrobacterium and other methods has been described. (Shuerman et al. (1993) Scientia Horticulturae 55: 101-124). For instance, regeneration and transformation of apples is described in James et al. (1989) Plant Cell Rep. 7: 658-661. Tissue culture procedures for apple including micropropagation, (Jones (1976) Nature 262: 392-393; Zimmerman (1983) Pp 124-135 In Methods in Fruit Breeding,) and adventitious bud formation (James (1987) Biotechnology and Genetic Engineering Reviews, 5: 33-79) have also been described. After transformation, transformed plant cells or plants comprising the introduced DNA must be identified. A selectable and/or scorable marker gene is typically used. However, the apoproteins can be detected directly through the formation of a fluorescent adduct with a bilin. In another embodiment, the apophytochrome (apoprotein) can be modified to utilize the endogenous or modified bilins produced in plants. Transformed plant cells can be selected by growing the cells on growth medium containing the appropriate antibiotic. In some instances, the presence of opines can also be used if the plants are transformed with Agrobacterium. After selecting the transformed cells, one can confirm expression of the introduced apoprotein gene(s). Simple detection of mRNA encoded by the inserted DNA can be achieved by well known methods in the art, such as Northern blot hybridization. The inserted sequence can be identified using the polymerase chain reaction (PCR) and Southern blot hybridization, as well (see, e.g., Sambrook, supra.).

Transformed plant cells (e.g., protoplasts) which are derived by any of the above transformation techniques can be cultured to regenerate a whole plant which possesses the transformed genotype and thus expresses the desired apoprotein. Such regeneration techniques rely on manipulation of certain phytohormones in a tissue culture growth medium. Plant regeneration from cultured protoplasts is described in Evans et al. (1983) pp. 124-176 In: Protoplasts Isolation and Culture, Handbook of Plant Cell Culture, MacMillan Publishing Company, New York; and Binding, Regeneration of Plants, Plant Protoplasts, pp. 21-73; CRC Press, Boca Raton, 1985. Regeneration can also be obtained from plant callus, explants, organs, or parts thereof. Such regeneration techniques are described generally in Klee et al. (1987) Ann. Rev. of Plant Phys. 38: 467-486.

One of skill will recognize that after the expression cassette is stably incorporated in transgenic plants and confirmed to be operable, it can be introduced into other plants by sexual crossing. Any of a number of standard breeding techniques can be used, depending upon the species to be crossed.

Expression in Mammalian and Insect Cell Cultures

Illustrative of cell cultures useful for the production of the apoprotein polypeptides are cells of insect or mammalian origin. Mammalian cell systems often will be in the form of monolayers of cells although mammalian cell suspensions may also be used. Illustrative examples of mammalian cell lines include VERO and HeLa cells, Chinese hamster ovary (CHO) cell lines, W138, BHK, Cos-7 or MDCK cell lines.

When the host cell is of insect or mammalian origin illustrative expression control sequences are obtained from the SV-40 promoter (Science, 222:524-527, 1983), the CMV I.E. Promoter (Proc. Natl. Acad. Sci. 81:659-663, 1984) or the metallothionein promoter (Nature 296:39-42, 1982). The cloning vector containing the expression control sequences is cleaved using restriction enzymes and adjusted in size as necessary or desirable and ligated with DNA coding for the apoprotein polypeptides by means well known in the art.

Expression of Variant Apoprotein Polypeptides

The nucleotide sequences used to transfect the host cells described above and used for production of recombinant binding domain polypeptides can be modified according to standard techniques to yield polypeptides with a variety of desired properties. The binding domain polypeptides of the present invention can be readily designed and manufactured utilizing various recombinant DNA techniques well known to those skilled in the art. For example, the binding domain polypeptides can vary from the naturally-occurring sequence at the primary structure level by amino acid insertions, substitutions, deletions, and the like. These modifications can be used in a number of combinations to produce the final modified protein chain.

The amino acid sequence variants can be prepared with various objectives in mind, including facilitating purification and preparation of the recombinant polypeptide, biological stability, and/or fluorescence quantum yields of the adducts of the invention.

In general, modifications of the sequences encoding the apoprotein polypeptides may be readily accomplished by a variety of well-known techniques, such as site-directed mutagenesis (see, Giliman and Smith (1979) Gene 8:81-97 and Roberts et al. (1987) Nature 328:731-734), or chemical modification (Glazer et al. (1975) Pp, 205 in Chemical Modification of Proteins, Elsevier, New York).

One of ordinary skill will appreciate that the effect of many mutations is difficult to predict. Thus, most modifications are evaluated by routine screening in a suitable assay for the desired characteristic. A particularly useful assay using expression in the yeast, Pichia pastoris is described below and in the examples. For instance, this assay can be used to test random genetic approaches to identify ‘gain-of-function’ mutations which affect the spectroscopic properties of phytochrome.

Fluorescence-based screens of the phytochrome mutant expressing cell population are particularly useful in the Pichia system because these cells synthesize PΦB (Wu et al (1996) Proc. Natl. Acad. Sci. USA, 93: 8989-8994). In this way, mutations affecting the primary photochemical step in the conversion of Pr to Pfr (i.e. 15Z to 15E photoisomerization) will exhibit enhanced fluorescence. Fluorescence-activated cell sorting (FACS) is particularly useful in this assay. The introduction of a bulky amino acid side chain near the D-ring of the chromophore is one example of the type of mutation which can be isolated by this screen.

Specific amino acid residues important to chromophore-protein interactions in phytochrome can be identified. For instance, epitope-tagged versions of recombinant phytochromes derived from higher plants (i.e. AsphyA-ST and AtphyA-ST), the green alga Mesotaenium caldariorum (i.e. Mcphyl1b-ST) and the cyanobacterium Synechocystis sp. PCC6803 (i.e. S6803phy1-ST)—all four of which have been successfully expressed and assembled with bilins can be used to identify useful variants.

Phytochromes can be used in these methods. The HPLC analyses are greatly simplified by the use of a chromophore domain fragment. The expression and purification of such mutants of AsphyA or Mcphy1b is based on chromophore domain mutant expression studies of other species (see, e.g., Deforce et al. (1991) Proc Natl Acad Sci USA 88:10392-10396 and Schmidt et al. (1996) J. Photochem. Photobiol., B: Biology 34: 73-77.

In one embodiment, a preferred apoprotein consists of the chromophore domain; the N terminus of the apoprotein sufficient for lyase activity. In a particularly preferred embodiment, the apoprotein consists of the minimal chromophore domain. Such minimal domains are readily determined by performing apoprotein truncations and assaying the ability of the aproprotein to reassemble with an added bilin as described herein. One such shortened apoprotein consists of 197 amino acids, described in the Examples.

Particular amino acid sites can also be modified. One such site is the chromophore binding site cysteine residue (i.e. cys₃₂₂ of AsphyA, cys₃₂₄ of Mcphy1b, or cys₂₅₉ of Cph1). These residues can be modified with a sulfhydryl-specific bifunctional photoaffinity crosslinking reagent such as p-azidophenacyl bromide or N(4-azidophenylthio)phthalimide (APTP). The bifunctional photoaffinity crosslinking reagent will be introduced into the molecule via reaction with cys₃₂₂ followed by UV crosslinklng. Having identified putative chromophore binding site residues with this approach, saturation site-specific mutagenesis experiments will be undertaken to evaluate the importance of these residues to bilin attachment, photoactivity and/or holoprotein conformation. Control experiments to determine whether chemical modification grossly alters the apoprotein's conformation will also be performed with each sulfhydryl reagent. Other residues can be modified as shown in Example 2. Some residues are required for bilin binding and therefore must be conserved in modified apoproteins of the invention. For instance, as shown in Example 2, modification of the glutamic acid residue at position 189 of Cph1 abolished bilin binding.

Based on multiple sequence alignment of the chromophore domains of phytochromes directed mutagenesis can be carried out. In one embodiment, a “chemical rescue” approach can be employed to help distinguish specific local effects from gross structural perturbations caused by individual mutations (see, Toney et al. (1989) Science 243:1485-1488). Using this technique, site-directed mutations at conserved arg and trp residues can be introduced within the chromophore domain of phytochrome. Arg₂₃₇ of AsphyA is an example of a good target for mutagenesis because it is the only conserved arg residue in the chromophore domain, and thus is a potential candidate for tethering the propionic acid side chains of the bilin chromophore.

A similar approach is used to examine the importance of conserved tryptophan residues, beginning with the two universally invariant trp₃₆₆ and trp₄₇₅ of AsphyA. In this case, chemical rescue will employ indole prosthesis. The potential importance of trp residues to the phytochrome photocycle has already been implicated by resonance Raman spectroscopy (see, e.g., Mizutani et al. (1991) Biochemistry 30:10693-10700).

Other cysteine residues in the chromophore domain can also be mutagenized. First, there are relatively few cysteine residues in phytochrome with as few as 6 cysteines in the chromophore domain of S6803phy1. In addition, aside from the site of chromophore attachment, only one other cysteine (i.e. cys₃₈₇ on AsphyA) is found on almost all of the known phytochromes, the notable exception being rcaE. This suggests that most, if not all of the cysteines are dispensable, and could be substituted with isosteric serine residues without any significant structural or functional effect. For instance, the five cysteine residues in the chromophore domain of S6803phy1 can be substituted with serine residues. The photochemical properties of these mutant constructs can be examined to ascertain if the absorption coefficient, photoequilibrium and/or photochemical quantum yields are altered by mutagenesis. Another preferred embodiment is a cysteine-deficient (except for cys₂₅₉ of S6803phy1), photoactive phytochrome mutant. This mutant is particularly useful for structural studies such as crosslinking experiments proposed above. Moreover, re-introduction of cysteine residues at selected positions in this cysteine-deficient mutant can be used for structural analyses, and for specific cross-linking to other macromolecules.

Preparation of bilins

The bilin component of the adducts of the invention can be isolated from the appropriate natural source or synthesized according to techniques known in the art. Methods for synthesis of the dimethyl ester of phytochromobilin are described for instance in Weller et al. (1980) Chem. Ber. 113:1603-1611. Conversion of the dimethyl ester to the free acid can be accomplished according to known techniques (see, e.g., Greene and Wuts, Protective Groups in Organic Synthesis 2d ed. (John Wiley and Sons, 1991).

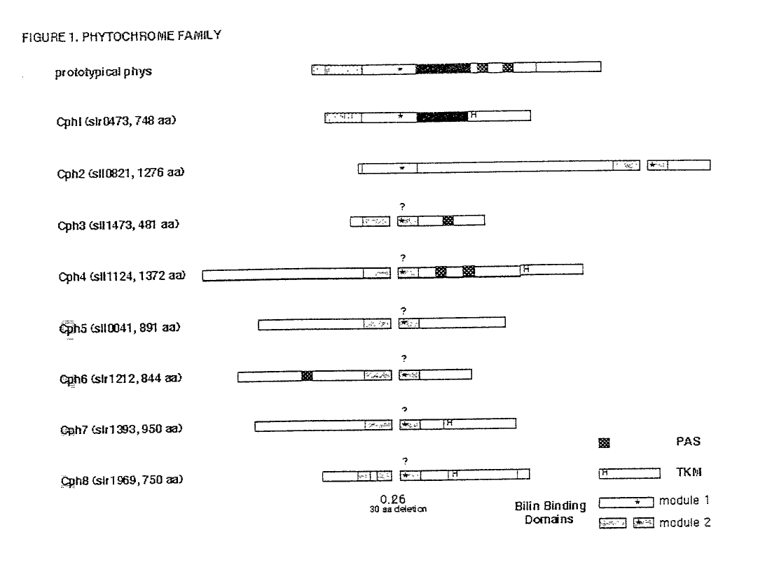

Methods for isolating bilins including phytochromobilin, phycocyanobilin (PCB), and phycoerythrobilin (PEB) from natural sources are also described in the art. For instance crude phycocyanobilin can be prepared from Spirulina platensis as described by Terry et al. (1993) J. Biol. Chem. 268:26099-26106. Crude phytochromobilin and PEB can be prepared by methanolysis of Porphyridium cruentum cells as described by Cornejo et al. (1992) J. Biol. Chem. 267: 14790-14798. The structures of phytochromobilin, PCB, and PEB are shown in FIG. 1 of WO 98/04700.

Attachment of fluorescent adducts to subject molecules.

Tagged moiety.

The conjugates of the subject invention are fluorescent adducts bound either covalently or non-covalently, normally covalently, to a particular moiety to be detected. Virtually any moiety to which it is desired to attach a fluorescent label is suitable. The moiety can be a macroscopic article such as an article of manufacture that is to be fluorescently tagged, or alternatively, the moiety can be microscopic, such as cell, an organelle, or a single molecule.

Again, virtually any molecule can be tagged. Typically, however, the moiety to be tagged and detected will be a biomolecule such as a polypeptide, oligopeptide, nucleic acid, polysaccharide, oligosaccharide, lipid, and the like. For instance, the subject molecule may be a ligand or receptor. A “ligand”, as used herein, refers generally to all molecules capable of reacting with or otherwise recognizing or binding to a second biological macromolecule e.g., a receptor, antigen, or other molecule on a target cell. Specifically, examples of ligands include, but are not limited to antibodies, lymphokines, cytokines, receptor proteins (e.g., CD4, CD8), solubilized receptor proteins (e.g., solubilized T-cell receptor, soluble CD4), hormones, growth factors, and the like which specifically bind particular target cells. A “growth factor” as used herein refers to a protein ligand that stimulates cell division or differentiation or inhibits cell division or stimulates or inhibits a biological response like motility or secretion of proteins. Growth factors are well known to those of skill in the art and include, but are not limited to, platelet-derived growth factor (PDGF), epidermal growth factor (EGF), insulin-like growth factor (IGF), transforming growth factor β (TGF-β), fibroblast growth factors (FGF), interleukin 2 (IL2), nerve growth factor (NGF), interleukin 3 (IL3), interleukin 4 (IL4), interleukin 1 (IL1), interleukin 6 (IL6),interleukin 7 (IL7), granulocyte/macrophage colony-stimulating factor (GM-CSF), granulocyte colony-stimulating factor (G-CSF), macrophage colony-stimulating factor (M-CSF), erythropoietin and the like. One of skill in the art recognizes that the term growth factor as used herein generally includes cytokines and colony stimulating factors.

Attachment of the phytofluor to the moiety.

The proteinaceous portions of the fluorescent adducts (phytofluors) referred to here as the apoproteins provide a wide range of functional groups for conjugation to proteinaceous and non-proteinaceous molecules. Functional groups which are present include, but are not limited to amino, thio, hydroxyl, and carboxy. In some instances, it may be desirable to introduce, delete, or modify functional groups, particularly thio groups where the apoprotein is to be conjugated to another protein.

Depending upon the nature of the molecule (e.g., member of a specific binding pair) to be conjugated to the phytofluor complex, the ratio of the two moieties will vary widely, where there may be a plurality of subject molecules to one phytofluor or apoprotein or, conversely, where there may be a plurality of phytofluors or apoproteins to one subject molecule. Of course, the molar ratio of the molecule (moiety) to be labeled to the phytofluor or apoprotein may be about 1:1. In addition, in some instances, initial intermediates are formed by covalently conjugating a small ligand to a fluorescent adduct and then forming a specific binding pair complex with the complementary receptor, where the receptor then serves as a ligand or receptor in a subsequent complex or is itself covalently attached to a ligand or receptor intended for use in a subsequent complex.

The procedure for attaching a subject molecule to the phytofluor or an apoprotein of the fluorescent adduct will vary according to the chemical structure of the agent. As indicated above, the apoproteins contain a variety of functional groups (e.g.,—OH,—COOH,—SH, or —NH₂) groups, which are available for reaction with a suitable functional group on an agent molecule to bind the agent thereto. Alternatively, the apoprotein may be derivatized to expose or attach additional reactive functional groups. The derivatization may involve attachment of any of a number of linker molecules such as those available from Pierce Chemical Company, Rockford Ill. A bifunctional linker having one functional group reactive with a group on a particular agent, and another group reactive with an antibody, may be used to form the desired immunoconjugate.

Alternatively, derivatization may involve chemical treatment of the antibody; e.g., glycol cleavage of the sugar moiety of the glycoprotein antibody with periodate to generate free aldehyde groups. The free aldehyde groups on the antibody may be reacted with free amine or hydrazine groups on an agent to bind the agent thereto (see, e.g., U.S. Pat. No. 4,671,958). Procedures for generation of free sulfhydryl groups on antibodies or antibody fragments are also known (see, e.g., U.S. Pat. No. 4,659,839). Many procedure and linker molecules for attachment of various compounds including radionuclide metal chelates, toxins and drugs to proteins (e.g., to antibodies) are known. See, for example, European Patent Application No. 188,256; U.S. Pat. Nos. 4,671,958, 4,659,839, 4,414,148, 4,699,784; 4,680,338; 4,569,789; and 4,589,071; and Borlinghaus et al. (1987) Cancer Res. 47: 4071-4075).

Linking agents suitable for joining the adducts of this invention to nucleic acids are also well known. For example, linking agents which are specific to the free secondary hydroxyl normally present at the 3′ end include phosphites, succinic anhydride and phthalamide. Linking agents which are specific to the phosphate normally present on the sugar at the 5′ end (at least for most naturally occurring polynucleotides or products of most cleavage reactions) include carbodiimides such as 1-ethyl-,3′dimethylamino propylcarbodiimide, with or without imidazole or 1-methylimidazole. See Chu et al. (1983) Nucleic Acids Res. 11: 6513-6529.

Use of apoproteins as affinity chromatography reagents

In addition to use in preparation of phytofluors, the apoproteins of the invention can be used in standard affinity chromatography methods to isolate desired compounds. For instance, as noted above, the apoproteins of the invention form a covalent bond with the bilin component of phytochromes and phytofluors, this interaction can be used to isolate and characterize novel bilins from plant materials. Thus, the apoproteins (e.g. those consisting of the chromophore domain) can be attached to a solid surface (e.g. beads) according to standard techniques (e.g. via a genetically engineered affinity peptide tag) and used as affinity chromatography reagent in standard isolation techniques.

Alternatively, nucleic acids encoding apoproteins can be used to prepare fusion proteins to identify proteins that interact with the fusion partner. By attaching a bilin to a solid matrix in an appropriate manner, chromophore domain-containing test proteins could be ‘captured’ enabling the isolation of proteins that interact with them.

Apoprotein-containing kits.

In one embodiment this invention provides kits utilizing the labels and/or binding domains of this invention. The kits preferably include one or more of the apoproteins of this invention and/or one or more nucleic acids encoding the apoproteins. Where the kits are intended for labeling, the kits can additionally include a one or more bilins that form fluorescent adduct(s) with the apoprotein, and/or appropriate reagents for coupling the apoprotein and/or bilin to the moiety that is to be labeled.

In another embodiment the kits can include nucleic acids encoding the apoprotein. The nucleic acid can be a nucleic acid vector containing appropriate restriction sites to faciliate insertion of a heterologous DNA such that the vector expresses the a heterologous polypeptide in fusion with the apoprotein.

In still another embodiment, the kits contain apoproteins for creation of an affinity column. In this instance, the kits can additionally include an affinity matrix to which the apoprotein can be bound, or the apoprotein can be provided already bound to a solid support (e.g. to polymeric beads or other materials).

The kits may optionally contain any of the buffers, reagents, and/or media that are useful for the practice of the methods of this invention.

In addition, the kits may include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the present invention.

### Example 1

### New Apophytochromes From Synechocystis

WO 98/04700 shows that incubation of phytochrome apoproteins with the linear tetrapyrrole phycoerythrin produces intensely fluorescent protein complexes known as phytofluors (see, also, Murphy et al Curr. Biol. 7:870-876 (1997). FIG. 6 of WO 98/04700 presents a sequence alignment of members of the apophytochrome family including eukaryotic members and one prokarotic member named S6803phy1 or cph1. This example shows that other apophytochromes genes are present in the cyanobacterium, Synechocystis sp. PCC 6803. The encoded polypeptides covalently associate with bilins to produce biliproteins both with phytochrome-like spectroscopic properties and others with significantly altered absorption spectra.

This example provides further data that define the chromophore-binding domain on the phytochrome ‘superfamily’ of related proteins which is required for covalent binding of the bilin prosthetic group (the chromophore domain). This chromophore domain has been delimited to a region roughly 200 amino acids in length.

The multiple sequence alignment shown in FIG. 6 of WO 98/04700 was used to construct a protein profile (as described by Gribskov, M. & Veretnik, S.: Identification of sequence pattern with profile analysis in Methods in Enzymology. 1996:198-212). for profilesearching of the Cyanobase cyanobacterial database (Cyanobase Web Site. http://www.kazusa.or.jp/cyano/).

Based upon this search, seven additional apophytochrome protein sequences (i.e. cph2-8) were identified—one of which (cph2) has two phytochrome-related domains. The genetic loci of these sequences in the genome of the cyanobacterium Synechocystis sp PCt6803 are shown in Table 1.

TABLE 1
Phytochrome-Related Sequences in the Genome of
Synechocystis sp PCC6803.

| Gene ID | Cyanobase Locus | Protein Length (aa) |
|---|---|---|
| Cph1 | SLR0473 | 748 aa |
| Cph2 | SLL0821 | 1276 aa |
| Cpl1 (Cph3) | SLL1473 | 481 aa |
| Cpl2 (Cph4) | SLL1124 | 1372 aa |
| Cpl3 (Cph5) | SLL0041 | 891 aa |
| Cpl4 (Cph6) | SLR1212 | 844 aa |
| Cpl5 (Cph7) | SLR1393 | 950 aa |
| Cpl6 (Cph8) | SLR1969 | 750 aa |

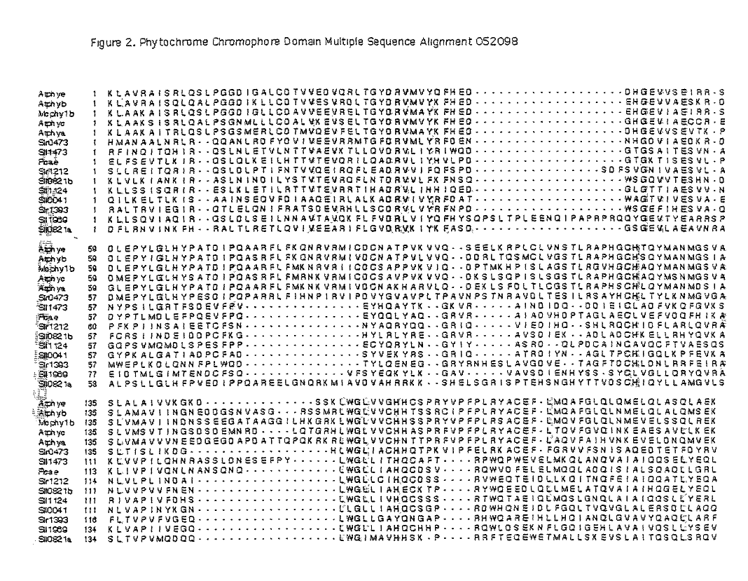

FIG. 1 shows the putative structural organization of the predicted protein products of these open reading frames. The roughly 200 amino acid chromophore domain of these proteins which include the cysteine site of bilin attachment is shown with an asterisk. Two major subfamilies have been defined—the first subfamily consisting of higher plant phytochrome-like domains, which include cph1, and the N-terminal domain of cph2 (i.e cph2a), and a second subfamily consisting of cp1 (phytochrome-like) 1-6 (also referred to as cph3-8) and the second domain of cph2 (i.e. cph2b) which have a roughly 30 amino acid deletion adjacent to the predicted cysteine site for bilin attachment. A multiple sequence alignment of the chromophore domains of representative eukaryotic phytochromes which include the Arabidopsis phyA, phyB/D, phyC and phyE proteins and the green algal phytochrome (Mcphy1b) and the cyanobacterial phytochrome-related sequences, shown in FIG. 2 was obtained using the Wisconsin Genetics Computing Group program PILEUP. This multiple sequence alignment defines the bilin chromophore binding domain of the phytochrome superfamily.

To demonstrate this, DNA fragments that encompass the chromophore-related domains of cph1, cph2a, cph2b and cph8 were isolated by PCR, cloned these into an expression vector, and tested as described in WO 98/04700. These experiments confirmed that cph1, cph1 (N514 deletion mutant), and cph2a (N390, consisting of the first 390 amino acid residues) all yield photochemically active phytochrome-like species upon incubation with PCB. Cph2a(N390), cph2b(C423) and cph8 polypeptides also covalently bind PCB. The PCB adducts of cph2b and cph 8 are both photochemically inactive and absorb mostly in the blue wavelength region. This shows that the cph2b/cph3/cph4/cph5/cph6/cph7/cph8 phytochrome ‘subfamily’ is able to catalyze bilin attachment and, like cph1/cph2a, they are apophytochromes. This work thus confirms that the structural motif illustrated in FIG. 2 represents the bilin-binding domain of the ‘phytochrome superfamily’ from which new phytofluors of different colors can be devised. Two subfamilies of cyanobacterial ‘phytochromes’ defined by cph1/2a and cph2b/cph3/cph4/cph5/cph6/cph7/cph8 which respectively exhibit red-far-red photoreversible and blue light absorbing non-photoreversible molecules have also been defined.

### Example 2

### Further Definition of the Chromophore Domain

This example presents experimental data demonstrating that a phytofluor can be produced by expression of a apoprotein polypeptides of only 197 amino acids in length. This result defines a roughly 200 amino acid bilin binding domain motif that is sufficient for covalent attachment of a bilin chromophore precursor to produce photoactive adducts (ie. phytochrome) and fluorescent adducts (ie. phytofluors). In addition, mutagenesis of some of the fully conversed residues within this domain of cph1 has confirmed the importance of these residues for bilin assembly and spectroscopic properties of the resulting bilin adducts.

Example 1 provides evidence of two class of apophytochromes. The first class possess a 200 amino acid protein motif termed ‘module 1’ that is similar to eukaryotic phytochromes. Members of this class include Cph1 and Cph2 (ie. a region encompassing the N-terminal 390 amino acids), that both yield red, far-red photoreversible phycocyanobilin (PCB) adducts. The second class, typified by Cph8 and the C-terminal 423 amino acid region of Cph2, afford ‘non-photoactive’ biliproteins upon incubation with PCB. Members of this class, possess a 200 amino acid domain (ie. module 2) that is distinguished from the first class by a conspicuous deletion of 17-22 amino acids near the cysteine attachment site.

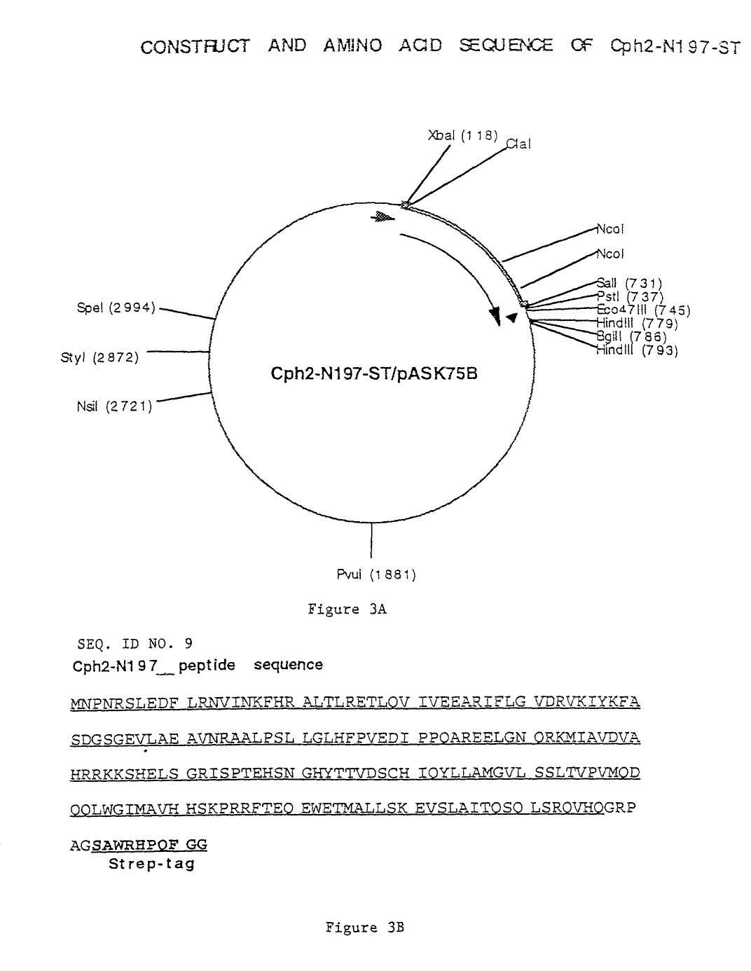

Based on the multiple sequence alignment shown in FIG. 2, and using methods described in W098/04700, a bacterial expression vector express a Strep-Tagged version of N-terminal 197 amino acid region of Cph2 (Cph2-N197) that encompassed ‘module 1’ only was constructed (see, FIG. 3). E. coli cells containing this plasmid produced the Strep-Tagged protein of the expected size (ie. 25 kDa), that yielded a covalent biliprotein adduct upon incubation with PCB as detected by zinc blot visualization Purification of the PCB-Cph2 adduct yielded a protein possessing phytochrome-like optical properties including a red minus far-red difference spectra. PEB treatment of extracts containing cph2-N197 also produced a fluorescent species with excitation and emission properties nearly indistinguishable from other phytofluors. Taken together, these experiment demonstrate that module 1 region of cph2 is competent to covalently bind bilins and therefore represents a functional chromophore domain.

To address the importance of conserved residues in the bilin lyase domain of Cph1 to bilin attachment and spectral properties of its bilin adducts, site-directed mutagenesis of some of these residues were undertaken. Charged residues D171, R172, E189 and R222 of Cph1 (N514) were mutagenized in the initial studies. The results of these experiments are summarized in Table 2. These experiments show that E189 is required for bilin binding, since its conversion to the amino acids A, G, K or T produces an apoprotein that is incapable of bilin binding. By contrast, single missense mutations of redisues D171, R172 and R222 did not abolish bilin binding, but instead lead to phytochromes with altered spectral properties. Double mutants of residues D171 and R172 also failed to bind bilin. Taken together, these experiments underscore the importance of these residues to the photosensory activity of phytochrome, and show that mutagenesis of the bilin lyase domain can be used for ‘tuning’ the photochemical and/or fluorescence properties of apophytochrome-bilin adducts.

TABLE 2
Bilin Binding and Photochemical Activity of PCB Adducts of Sited-
Directed Mutants of Cph1 (N514).

| | PCB | | Difference |
|---|---|---|---|
| | Binding | Photoreveribility | Spectrum |
| Single Mutants | | | |
| Cph1 (N514) Wild Type | yes | yes | WT |
| Cph1 (N514) D171A | yes | yes | WT |
| Cph1 (N514) | yes | yes | blue shifted |
| R172G/A | | | |
| Cph1 (N514) E189A, | no | no | none |
| G K or T | | | |
| Cph1 (N514) R222G | yes | yes | blue shifted |
| Double Mutants | | | |
| Cph1 (N514) | no | no | none |
| DR171/2AA | | | |
| Cph1 (N514) | no | no | none |
| DR171/2AG | | | |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

What is claimed is:

1. A composition comprising an apoprotein polypeptide of between about 190 amino acids and about 400 amino acids, which apoprotein polypeptide comprises a lyase domain.

2. The composition of claim 1, wherein the apoprotein polypeptide is selected from the group consisting of a plant apoprotein, an algal apoprotein, and a cyanobacterial apoprotein.

3. The composition of claim 1, wherein the apoprotein polypeptide consists of about 390 amino acids.

4. The composition of claim 1, wherein the apoprotein polypeptide consists of about 200 amino acids.

5. The composition of claim 4, wherein the aopoprotein protein is as shown in SEQ ID NO: 9.

6. The composition of claim 1, wherein the apoprotein protein consists of a lyase domain.

7. The composition of claim 1, wherein the apoprotein polypeptide is from Synechocystis sp.

8. The composition of claim 7, wherein the apoprotein polypeptide is Cph2.

9. The composition of claim 1, wherein the apoprotein is covalently linked to a bilin to form a fluorescent adduct.

10. The composition of claim 9, wherein the bilin is a tetrapyrrole.

11. The composition of claim 10, wherein the bilin is phycoerythrobilin.

12. The composition of claim 9, wherein the fluorescent adduct is linked to a biomolecule.

13. The composition of claim 12, wherein the biomolecule is selected from the group consisting of a protein, a carbohydrate, a lipid, and a nucleic acid.

14. The composition of claim 13, wherein the biomolecule is a nucleic acid.

15. The composition of claim 13, wherein the biomolecule is a protein.

16. The composition of claim 15, wherein the protein is an antibody.

17. A method of detecting the presence of a biomolecule in a sample, the method comprising:
providing a sample comprising a biomolecule linked to a fluorescent adduct consisting of a bilin and an apoprotein of between about 190 amino acids and about 400 amino acids, which apoprotein polypeptide comprises a lyase domain;
contacting the sample with light which causes the fluorescent adduct to emit light;
detecting the emitted light, thereby detecting the presence of the biomolecule.

18. The method of claim 17, wherein the step of contacting the sample with light includes contacting the sample with light having a wavelength of about 570 nm.

19. The method of claim 17, wherein the step of detecting the emitted light includes detecting light having a wavelength of about 590 nm.

20. The method of claim 17, wherein the apoprotein polypeptide is selected from the group consisting of a plant apoprotein, an algal apoprotein, and a cyanobacterial apoprotein.

21. The method of claim 17, wherein the apoprotein polypeptide consists of a lyase domain.

22. The method of claim 17, wherein the apoprotein polypeptide consists of about 390 amino acids.

23. The method of claim 17, wherein the apoprotein polypeptide consists of about 200 amino acids.

24. The method of claim 23, wherein the aopoprotein is a shown in SEQ ID NO: 9.

25. The method of claim 17, wherein the apoprotein polypeptide is from Synechocystis sp.

26. The method of claim 25, wherein the apoprotein polypeptide is Cph2.

27. The method of claim 17, wherein the bilin is a tetrapyrrole.

28. The method of claim 27, wherein the bilin is phycoerythrobilin.

29. The method of claim 17, wherein the biomolecule is selected from the group consisting of a protein, a carbohydrate, a lipid, and a nucleic acid.

30. The method of claim 29, wherein the biomolecule is a nucleic acid.

31. The method of claim 29, wherein the biomolecule is a protein.

32. The method of claim 31, wherein the protein is an antibody.


SEQUENCE LISTING (24 sequences)

SEQ ID NO: 1 - protein, 748 aa, Unknown
  Description of Unknown Organism Locus SLR0473 = S6803PHY1 = SYN_PHY
MATTVQLSDQSLRQLETLAIHTAHLIQPHGLVVVLQEPDLTISQISANCTGILGRSPEDLLGRTLGEVFDSFQIDPIQSRLTAGQISSLNPSKLWARVMGDDFVIFDGVFHRNSDGLLVCELEPAYTSDNLPFLGFYHMANAALNRLRQQANLRDFYDVIVEEVRRMTGFDRVMLYRFDENNHGDVIAEDKRDDMEPYLGLHYPESDIPQPARRLFIHNPIRVIPDVYGVAVPLTPAVNPSTNRAVDLTESILRSAYHCHLTYLKNMGVGASLTISLIKDGHLWGLIACHHQTPKVIPFELRKACEFFGRVVFSNISAQEDTETFDYRVQLAEHEAVLLDKMTTAADFVEGLTNHPDRLLGLTGSQGAAICFGEKLILVGETPDEKAVQYLLQWLENREVQDVFFTSSLSQIYPDAVNFKSVASGLLAIPIARHNFLLWFRPEVLQTVNWGGDPNHAYEATQEDGKIELHPRQSFDLWKEIVRLQSLPWQSVEIQSALALKKAIVNLILRQAEELAQLARNLERSNADLKKFAYIASHDLQEPLNQVSNYVQLLEMRYSEALDEDAKDFIDFAVTGVSLMQTLIDDILTYAKVDTQYAQLTFTDVQEVVDKALANLKQRIEESGAEIEVGSMPAVMADQIQLMQVFQNLIANGIKFAGDKSPKIKIWGDRQEDAWVFAVQDNGIGIDPQFFERIFVIFQRLHTRDEYKGTGMGLAICKKIIEGHQGQIWLESNPGEGSTFYFSIPIGN

SEQ ID NO: 2 - protein, 1276 aa, Unknown
  Description of Unknown Organismcph2 Locus SLL0821
MNPNRSLEDFLRNVINKFHRALTLRETLQVIVEEARIFLGVDRVKIYKFASDGSGEVLAEAVNRAALPSLLGLHFPVEDIPPQAREELGNQRKMIAVDVAHRRKKSHELSGRISPTEHSNGHYTTVDSCHIQYLLAMGVLSSLTVPVMQDQQLWGIMAVHHSKPRRFTEQEWETMALLSKEVSLAITQSQLSRQVHQQQVQEALVQRLETTVAQYGDRPETWQYALETVGQAVEADGAVLYIAPDLTGSVAQHYQWNLRFDWGNWLETSLWQELMRGQPSAAMEPMAAVQSTWEKPRPFTSVAPLPPTNCVPHGYTLGELEQRSDWIAPPESLSAENFQSFLIVPLAADQQWVGSLILLRKEKSLVKHWAGKRGIDRRNILPRLSFEAWEETQKLVPTWNRSERKLAQVASTQLYMAITQQFVTRLITQQTAYDPLTQLPNWIIFNRQLTLALLDALYEGKMVGVLVIAMDRFKRINESFGHKTGDGLLQEVADRLNQKLSPLAAYSPLLSRWHGDGFTILLTQISDNQEMIPLCERLLSTFQEPFFLQGQPIYLTASMGISTAPYDGETAESLLKFAEIALTRAKCQGKNTYQFYRPQDSAPMLDRLTLESDLRQALTNQEFVLYFQPQVALDTGKLLGVEALVRWQHPRLGQVAPDVFIPLAEELGLINHLGQWVLETACATHQHFFRETGRRLRMAVNISARQFQDEKWLNSVLECLKRTGMPPEDLELEITESLMMEDIKGTVVLLHRLREEGVQVAIDDFGTGYSSLSILKQLPIHRLKIDKSFVNDLLNEGADTAIIQYVIDLANGLNLETVAEGIESEAQLQRLQKMGCHLGQGYFLTRPLPAEAMMTYLYYPQILDFGPTPPLPKVALPETETEAGQGNVGDRPLPNSLNRENPWTEKLHDYVLLKERLQQRNVKEKLVLKIANKIRASLNINDILYSTVTEVRQFLNTDRVVLFKFNSQWSGQVVTESHNDFCRSIINDEIDDPCFKGHYLRLYREGRVRAVSDIEKADLADCHKELLRHYQVKANLVVPVVFNENLWGLLIAHECKTPRYWQEEDLQLLMELATQVAIAIHQGELYEQLETANIRLQQISSLDALTQVGNRYLFDSTLEREWQRLQRIREPLALLLCDVDFFKGFNDNYGHPAGDRCLKKIADAMAKVAKRPTDLVARYGGEEFAIILSETSLEGAINVTEALQVEVANLAIPHTVSGTGHVTLSIGIAVYTPERHINPNALVKAADLALYEAKAKGRNQWLAYEGSQLPHVDGEV

SEQ ID NO: 3 - protein, 481 aa, Unknown
  Description of Unknown Organismcph Lucus SLL1473 a 297 aa histidine kinase homolog
MGKFLIPIEFVFLAIAMTCYLWHRQNQERRRIEISIKQQTQRERFINQITQHIRQSLNLETVLNTTVAEVKTLLQVDRVLIYRIWQDGTGSAITESVNANYPSILGRTFSDEVFPVEYHQAYTKGKVRAINDIDQDDIEICLADFVKQFGVKSKLVVPILQHNRASSLDNESEFPYLWGLLITHQCAFTRPWQPWEVELMKQLANQVAIAIQQSELYEQLQQLNKDLENRVEKRTQQLAATNQSLRMEISERQKTEAALRHTNHTLQSLIAASPRGIFTLNLADQIQIWNPTAERIFGWTETEIIAHPELLTSNILLEDYQQFKQKVLSGMVSPSLELKCQKKDGSWIEIVLSAAPLLDSEENIAGLVAVVADITEQKRQAEQIRLLQSVVVNTNDAVVITEAEPIDDPGPRILYVNEAFTKITGYTAEEMLGKTPRVLQGPKTSRTELDRVRQAISQWQSVTVEAEVLNDSYKEKKSPLK

SEQ ID NO: 4 - protein, 1371 aa, Unknown
  Description of Unknown Organismcph4 locus SLL1124 (DivJ homolog PAS domain) a 1372 aa protein that is more similar to rcaE than to cph1
MTFAATPREVTASAIQWACLCLPGELSAAEALNRWHRHGQRSWEPPAEAKAFPPWALVLDNDGQLLGLLPDWQLAAALWTEHFSPAIALAELCLPCSLRLDLEKLPSLGEVMQIFATWGYGWDVIPVADRQHQTWGLLSIGNLIRSVNLCQLWQNLPLQVTASPPLCLGTETTLGELVHHCFERQISSFPVVYSSPLLPAAAPRIPLGNVSLSNYFKGPNYGSLGLDNPIGPDLSPTFPLCTINQTYCHARELLRRQNDDYVIITNISGAFVGWVGPQQWLATVQPDVLLEALQREVEMPRIVQHLEARIVWQQQQQQRNQHLIQKLLSRNPNLIYLYDLVKNEIVYLNIPGSLLEGGSGGAPIPNPMVETDPRQDLLLPPRYFGLEELAALQAHEKKEFNFEFTDGGQSVHYFVVEISAFEIDGSGQTSKILCLAQDVSHGKRAEAALHTKEQQLQTLVNTIADGIVILDNHDKVIYANPMACQMFGLSKEEFLQSQLGLSNRGQTEIGINVSPEEEGIGEIKAVPIHWQGEDCRLVTVRDVTDRQRVLKKLRDSEQIHRSLLEALPNLVWRLSSAGDVWECNQRTLAYFGRRGRKILGNTWQQFIEPGERENVQRQWRQGIAAQEFFQLECRLWRSDGQYRWHLLQVLPLEDRFGSINGWLASSTDIDDLKEAEKALRNQAQQEKLLSSISQRIRESLKLETILRTTVTEVRRTIHADRVLIHHIQEDGLGTTIAESVVNGQPSVMQMDLSPESFPPECYQRYLNGYIYASRDQLPDCAINCAVQCFTVAESQSRIVAPIVFDHSLWGLLIVHQCSSSRTWQTAEIQLMQSLGNQLAIAIQQSLLYERLQEELSERQRAEQKLLEVNQLQKGIFDVANYMIISTDRRGIISTFNRTAEEILGYTAAELIGQQTPLIFHDQEEMASEAVQLSQQLQQTIRPNSIDMFAIPAIQWGVYEREWTYITKTGDRLPVYVSITALRDDQGKVDGLVGVITDLRRQKQIERERQNLDFVVKNSTELIVITDLEQKVTFLNQAGQSLIGLENPETAQTTYLSEHISPEYLNFWQMEIIPQVFRSGAWEGEFSLQHYQTAVEIPVTASVFLLQGVNGQHPANLVAIVHDITHIKNAEKRILAALEAEKELGELRSRFISTTSHEFRTPLAIISSSTGILKKYWPKLDGQRRGQHLERIEESVHHMVELLDDVLTINRAETKYLPFEPQPLDLVSFCRGITDELQSSTEYHGLLFSYDGLGPGEIVAFDPKLLRQILTNLLGNAIKYSPSGQPVEFHLQRRGDVGIFSVQDHGIGIGPEDIPNLFDSFYRGTNVGSIPGTGLGLPIVKKCAELHGGMITVTSQLGQGSRFEVELPLWYS

SEQ ID NO: 5 - protein, 891 aa, Unknown
  Description of Unknown Organismcph5 locus SLL0041 (locus 1001300) an 891 aa protein, methyl-accepting chemotaxis protein I.  Homology to tsr in last 250 aa.
MAEAFIAENTAVEDVSPNPNPAIDTDALAALTQSAVELTPPPPINLPKVELPPMQPLAPLMAIADPDNLSPMSTSIQAPTQSGGLSLRNKAVLIALLIGLIPAGVIGGLNLSSVDRLPVPQTEQQVKDSTTKQIRDQILIGLLVTAVGAAFVAYWMVGENTKAQTALALKAKHSHRNLDQPLAVAGDELAIADQTIDALSAQVEKLRHQQDLSLKQAELLTELSRANLSDIDEIQGVIQKNLDQARALFGCERLVFYYHPRYQPEAMVVQALDLTTQGLIDSKDPHPWGQEDMPSQIVAINDTSGASISNPHRQWLEQHQVKASLTVPLHRDNYPLGLLMAHHCQRPHQWEMRERQFLQQLTEELQTTLDRANLIQERNESAQQAQILKELTLKISAAINSEQVFDIAAQEIRLALKADRVIVYRFDATWAGTVIVESVAEGYPKALGATIADPCFADSYVEKYRSGRIQATRDIYNAGLTPCHIGQLKPFEVKANLVAPINYKGNLLGLLIAHQCSGPRDWHQNEIDLFGQLTVQVGLALERSDLLAQQKIAEVEQRQMREKMQKRALELLMEVDPVSRGDLTIRAHVTEDEIGTIADSYNATIESLRRIVTQVQTAASQFTETTDTNEVAVRQLAQQANRQALDVAEALERLQAMNKSIQAVAENAAQAESAVQRATQTVDQGEDAMNRTVDGIVAIRETVAATAKQVKRLGESSQKISKVVNLIGSFADQTNLLALNAAIEAAHAGEEGRGFAVVADEVRSLARQSAEATAEIAQLVATIQAETNEVVNAMEAGTEQVVVGTKLVEETRRSLNQITAVSAQISGLVEAITSAAIEQSQTSESVTQTMALVAQIADKNSSEASGVSATFKELLAVAQSLQEAVKQFKVQ

SEQ ID NO: 6 - protein, 844 aa, Unknown
  Description of Unknown Organismcph6 locus SLR12112 (ETR1 homolog; PAS domain) an 844 aa protein.  Chromophore domain 461-628.
MAITAFTLGDFFQANSYIPHGHCYLWQTPLVWLHVSADFFTAIAYYSIPLTLLYFLRKRQDIPFPNIIFLFSTFILCCGTSHFFDIITLWYPIYWISGTVKASMAIVSIITVFELIQIVPNALNLKSPTELATLNLALNQEIKERQTAEIALQELNNNLEKRVEDRTTQLAKINQQLEQEIEDKTRAKEDLEKNKDQLAQLAAIVESSQDAIISKTLDGNITSWNESAERLFGYTAEEMIGSHITKLIPEELILEEDLIAECIRQGQRINTYETQRQRKDGTKIDVALTISPIRDEHKNVVGASKIVRDITARLDVENALRESQYFIEKLANYSPQILYILDPIAWKNIYVNYQSFEILGYTPEEFKNGGTELLLNIVHPDDIPTLYENKNFWQKSQEGQVLTTEYRMRHKNGSWRWLRSREVVFARDDYGQVTKVLGTAQDISDSKEQEQRLYEQGRRESLLREITQRIRQSLDLPTIFNTVVQEIRQFLEADRVVIFQFSPDSDFSVGNIVAESVLAPFKPIINSAIEETCFSNNYAQRYQQGRIQVIEDIHQSHLRQCHIDFLARLQVRANLVLPLINDAILWGLLCIHQCDSSRVWEQTEIDLLKQITNQFEIAIQQATLYEQAQQELASKNQLFVQLTNELEQKKVLLKEIHHRVKNNLQIMSSLLYLQFSKASPAIQQLSEEYQNRIQSMALIHEQLYRSEDLANIDFSQYLKNLTHNICQSYGCNTDSIKIKLLVEQVKVPLEQSIPLGLIIQELVSNALKHAFPTTEGEISIKFTSMNSHYSLQVWDNGVGISRDIDLENTDSLGMQLIYSLTEQLQGELHYEYVGGAQFGLEFSL

SEQ ID NO: 7 - protein, 950 aa, Unknown
  Description of Unknown Organismcoh7 (locus SLR 1393) a 950 aa protein.  Chromophore domain 402-620.  Contains a histidine kinase transmitter domain.
MSPSSHGTAVQQAIADQLLEMILQSQDLHNAYRLVVEGLQRGLGVDRVLLVQNAVFPNRQSRLVAQAIAPARDIMLLDEPCADCRWLHLLGQLPHYGLWTVWEGEGEFVQLDPVQGEFCRTLGIKSLLHLPLVINQRHWGVLSLQYLHQARPWPLEDQQFAQRIAHLFCLGLMKTELWIHCQNHKNALQTVVAEGQVQRETYLKSAQRERAIADVIDKIRFALDLRSLFQTTVTEVRKLLVADRVMIIKVRQNKNFSWGEIQAEAQTDDKLCLLPPKERVPLSSRWIDHFAKGLILAMDDTDDQRADFDQSMLALAKANLVVPLFSGDRLWGVLSVHQCDGPRVWESSDIEFALKIALNLGVALQQAELLTESQRRSTALQSALGEVEAQKDYLARIAEEERALTRVIEGIRQTLELQNIFRATSDEVRHLLSCDRVLVYRFNPDWSGEFIHESVAQMWEPLKDLQNNFPLWQDTYLQENEGGRYRNHESLAVGDVETAGFTDCHLDNLRRFEIRAFLTVPVFVGEQLWGLLGAYQNGAPRHWQAREIHLLHQIANQLGVAVYQAQLLARFQEQSKTMENTLADLTAIVDNLADGLLVIDLFGRITRYNPALLAMFDLEGLELLGAGVDAYFPETLNQLLAKPEREEQKLVTADVELSQGRQGQALITSITSHENGCEYPQCLGAVIMIRDVTHEREVERMKTDFLATVSHELRTPLTSILGFATVIQDKLNRVIIPELDLAQPHLGKATERVMRNLAIIESEAQRLTVLINDVLDIAKMEAGQESWQEQPCAIGPIIERAIATITPQAQKKNISLQGDLEPGLPDFIGDENRILQVVLNLLSNAVKFTPKGLITARSHFHQNYLWVEIIDHGPGIHPADQEKIFEPFQQGGGDVLTDKPQGTGLGLPICKKIVEHHGGTIGVNSSLGRGSTFYFSLPVPVPAVETSPAV

SEQ ID NO: 8 - protein, 750 aa, Unknown
  Description of Unknown Organismcph8 (locus SLR1969) A 750 aa protein.  Chromophore domain 156-347.  Contains a histidine kinase transmitter domain.
MLPAFSPIFRRLLPAVTFERLLRFWRTLAQQTGDGVQCFVGDLPSSLKPPPGPSVLEAEVDHRFALLVSPGQWALLEGEQISPHHYAVSITFAQGIIEDFIQKQNLPVVAEAMPHRPETPSGPTIAEQLTLGLLEILNSDSTSFSPEPSLQDSLQASQVKLLSQVIAQIRQSLDLSEILNNAVTAVQKFLFVDRLVIYQFHYSQPSLTPLEENQIPAPRPRQQYGEVTYEARRSPEIDTMLGIMTENDCFSQVFSYEQKYLKGAVVAVSDIENHYSSSYCLVGLLQRYQVRAKLVAPIIVEGQLWGLLIAHQCHHPRQWLDSEKNFLGQIGEHLAVAIVQSLLYSEVQKQKNNFEKRVIERTKELRDTLMAAQAANLLKSQFINNISHELRTPLTSIIGLSATLLRWFDHPASLPPAKQQYYLLNIQENGKKLLDQINSIIQLSQLESGQTALNCQSFSLHTLAQTVIHSLLGVAIKQQINLELDYQINVGQDQFCADQERLDQILTQLLNNALKFTPAEGTVILRIWKESNQAIFQVEDTGIGINEQQLPVLFEAFKVAGDSYTSFYETGGVGLALTKQLVELHGGYIEVESSPGQGTIFTTVIPQQNFPPTTKGQVQDKLDAAMPFNSSVIVIEQDEEIATLICELLTVANYQVIWLIDTTNALQQVELLQPGLIIVDGDFVDVTEVTRGIKKSRRISKVTVFLLSESLSSAEWQALSQKGIDDYLLKPLQPELLLQRVQSIQQEPLR

SEQ ID NO: 9 - protein, 196 aa, Unknown
  Description of Unknown OrganismAtphye
KLAVRAISRLQSLPGGDIGALCDTVVEDVQRLTGYDRVMVYQFHEDDHGEVVSEIRRSDLEPYLGLHYPATDIPQAARFLFKQNRVRMICDCNATPVKVVQSEELKRPLCLVNSTLRAPHGCHTQYMANMGSVASLALAIVVKGKDSSKLWGLVVGHHCSPRYVPFPLRYACEFLMQAFGLQLQMELQLASQLAEK

SEQ ID NO: 10 - protein, 207 aa, Unknown
  Description of Unknown OrganismAtphyb
KLAVRAISQLQALPGGDIKLLCDTVVESVRDLTGYDRVMVYKFHEDEHGEVVAESKRDDLEPYIGLHYPATDIPQASRFLFKQNRVRMIVDCNATPVLVVQDDRLTQSMCLVGSTLRAPHGCHSQYMANMGSIASLAMAVIINGNEDDGSNVASGRSSMRLWGLVVCHHTSSRCIPFPLRYACEFLMQAFGLQLNMELQLALQMSEK

SEQ ID NO: 11 - protein, 210 aa, Unknown
  Description of Unknown OrganismMcphy1b
KLAAKAISRLQSLPGGDIGLLCDAVVEEVRELTGYDRVMAYKFHEDEHGEVIAEIRRSDLEPYLGLHYPATDIPQAARFLFMKNRVRIICDCSAPPVKVIQDPTMKHPISLAGSTLRGVHGCHAQYMANMGSVASLVMAVIINDNSSEEGATAAGGILHKGRKLWGLVVCHHSSPRYVPFPLRSACEFLMQVFGLQLNMEVELSSQLREK

SEQ ID NO: 12 - protein, 206 aa, Unknown
  Description of Unknown OrganismAtphyc
KLAAKSISRLQALPSGNMLLLCDALVKEVSELTGYDRVMVYKFHEDGHGEVIAECCREDMEPYLGLHYSATDIPQASRFLFMRNKVRMICDCSAVPVKVVQDKSLSQPISLSGSTLRAPHGCHAQYMSNMGSVASLVMSVTINGSDSDEMNRDLQTGRHLWGLVVCHHASPRFVPFPLRYACEFLTQVFGVQINKEAESAVLLKEK

SEQ ID NO: 13 - protein, 210 aa, Unknown
  Description of Unknown OrganismAtphya
KLAAKAITRLQSLPSGSMERLCDTMVQEVFELTGYDRVMAYKFHEDDHGEVVSEVTKPGLEPYLGLHYPATDIPQAARFLFMKNKVRMIVDCNAKHARVLQDEKLSFDLTLCGSTLRAPHSCHLQYMANMDSIASLVMAVVVNEEDGEGDAPDATTQPQKRKRLWGLVVCHNTTPRFVPFPLRYACEFLAQVFAIHVNKEVELDNQMVEK

SEQ ID NO: 14 - protein, 192 aa, Unknown
  Description of Unknown Organismslr0473
HMANAALNRLRQQANLRDFYDVIVEEVRRMTGFDRVMLYRFDENNHGDVIAEDKRDDMEPYLGLHYPESDIPQPARRLFIHNPIRVIPDVYGVAVPLTPAVNPSTNRAVDLTESILRSAYHCHLTYLKNMGVGASLTISLIKDGHLWGLIACHHQTPKVIPFELRKACEFFGRVVFSNISAQEDTETFDYRV

SEQ ID NO: 15 - protein, 177 aa, Unknown
  Description of Unknown Organismsl111473
RFINQITQHIRQSLNLETVLNTTVAEVKTLLQVDRVLIYRIWQDGTGSAITESVNANYPSILGRTFSDEVFPVEYHQAYTKGKVRAINDIDQDDIEICLADFVKQFGVKSKLVVPILQHNRASSLDNESEFPYLWGLLITHQCAFTRPWQPWEVELMKQLANQVAIAIQQSELYEQL

SEQ ID NO: 16 - protein, 173 aa, Unknown
  Description of Unknown OrganismRcae
ELFSEVTLKIRQSLQLKEILHTTVTEVQRILQADRVLIYHVLPDGTGKTISESVLPDYPTLMDLEFPQEVFPQEYQQLYAQGRVRAIADVHDPTAGLAECLVEFVDQFHIKAKLIVPIVQNLNANSQNQLWGLLIAHQCDSVRQWVDFELELMQQLADQISIALSQAQLLGRL

SEQ ID NO: 17 - protein, 168 aa, Unknown
  Description of Unknown Organismslr1212
SLLREITQRIRQSLDLPTIFNTVVQEIRQFLEADRVVIFQFSPDSDFSVGNIVAESVLAPFKPIINSAIEETCFSNNYAQRYQQGRIQVIEDIHQSHLRQCHIDFLARLQVRANLVLPLINDAILWGLLCIHQCDSSRVWEQTEIDLLKQITNQFEIAIQQATLYEQA

SEQ ID NO: 18 - protein, 165 aa, Unknown
  Description of Unknown Organismsl110821b
KLVLKIANKIRASLNINDILYSTVTEVRQFLNTDRVVLFKFNSQWSGQVVTESHNDFCRSIINDEIDDPCFKGHYLRLYREGRVRAVSDIEKADLADCHKELLRHYQVKANLVVPVVFNENLWGLLIAHECKTPRYWQEEDLQLLMELATQVAIAIHQGELYEQL

SEQ ID NO: 19 - protein, 165 aa, Unknown
  Description of Unknown Organismsl111124
KLLSSISQRIRESLKLETILRTTVTEVRRTIHADRVLIHHIQEDGLGTTIAESVVNGQPSVMQMDLSPESFPPECYQRYLNGYIYASRDQLPDCAINCAVQCFTVAESQSRIVAPIVFDHSLWGLLIVHQCSSSRTWQTAEIQLMQSLGNQLAIAIQQSLLYERL

SEQ ID NO: 20 - protein, 165 aa, Unknown
  Description of Unknown Organismsl110041
QILKELTLKISAAINSEQVFDIAAQEIRLALKADRVIVYRFDATWAGTVIVESVAEGYPKALGATIADPCFADSYVEKYRSGRIQATRDIYNAGLTPCHIGQLKPFEVKANLVAPINYKGNLLGLLIAHQCSGPRDWHQNEIDLFGQLTVQVGLALERSDLLAQQ

SEQ ID NO: 21 - protein, 170 aa, Unknown
  Description of Unknown Organismslr1393
RALTRVIEGIRQTLELQNIFRATSDEVRHLLSCDRVLVYRFNPDWSGEFIHESVAQMWEPLKDLQNNFPLWQDTYLQENEGGRYRNHESLAVGDVETAGFTDCHLDNLRRFEIRAFLTVPVFVGEQLWGLLGAYQNGAPRHWQAREIHLLHQIANQLGVAVYQAQLLARF

SEQ ID NO: 22 - protein, 188 aa, Unknown
  Description of Unknown Organismsl111969
KLLSQVIAQIRQSLDLSEILNNAVTAVQKFLFVDRLVIYQFHYSQPSLTPLEENQIPAPRPRQQYGEVTYEARRSPEIDTMLGIMTENDCFSQVFSYEQKYLKGAVVAVSDIENHYSSSYCLVGLLQRYQVRAKLVAPIIVEGQLWGLLIAHQCHHPRQWLDSEKNFLGQIGEHLAVAIVQSLLYSEV

SEQ ID NO: 23 - protein, 187 aa, Unknown
  Description of Unknown Organismsl110821a
DFLRNVINKFHRALTLRETLQVIVEEARIFLGVDRVKIYKFASDGSGEVLAEAVNRAALPSLLGLHFPVEDIPPQAREELGNQRKMIAVDVAHRRKKSHELSGRISPTEHSNGHYTTVDSCHIQYLLAMGVLSSLTVPVMQDQQLWGIMAVHHSKPRRFTEQEWETMALLSKEVSLAITQSQLSRQV

SEQ ID NO: 24 - protein, 210 aa, Artificial Sequence
  Description of Artificial SequenceCph2-N197
MNPNRSLEDFLRNVINKFHRALTLRETLQVIVEEARIFLGVDRVKIYKFASDGSGEVLAEAVNRAALPSLGLHFPVEDIPPQAREELGNQRKMIAVDVAHRRKKSHELSGRISPTEHSNGHYTTVDSCHIQYLLAMGVLSLTVPVMQDQQLWGIMAVHHSKPRRFTEQEWETMALLSKEVSLAITQSQLSRQVHQGRPAGSAWRHPQFGG